(19) United States
(12) Patent Application Publication
(10) Pub. No.: US 2026/0285789 A1
(43) Pub. Date: Sep. 24, 2026

(54) SELECTIVE SEMI-HYDROGENATION OF ALLENE

(71) Applicant: FIRMENICH SA, SATIGNY (CH)

(72) Inventor: Lionel Saudan, Satigny (CH)

(21) Appl. No.: 19/480,288

(22) PCT Filed: May 3, 2024
(86) PCT No.: PCT/EP2024/062226

§ 371 Date: Oct. 30, 2025

(30) Foreign Application Priority Data
May 3, 2023 (EP) 23171272.0

(51) Int. Cl. C07C 29/17; C07C 33/02; C07C 33/28

(52) U.S. Cl. CPC C07C 29/17; C07C 33/02; C07C 33/28

(57) ABSTRACT

The present invention relates to the field of catalytic hydrogenation and, more particularly, to the semi-hydrogenation of an allene into the corresponding alkene in the presence of a heterogenous catalyst.

### TECHNICAL FIELD

The present invention relates to the field of catalytic hydrogenation and, more particularly, to the semi-hydrogenation of an allene into the corresponding alkene in the presence of a heterogenous catalyst.

### BACKGROUND

Compounds comprising alkene, in particular ω-1 alkene group represent skeletons highly desirables which could be used as such or as key intermediates useful to prepare more complex compounds in different fields such as, among others, perfumery, cosmetic, pharmaceutic or agrochemistry. Said alkene group may be obtained by the reduction of the corresponding triple bond, method suitable in order to favour the formation of the double bond with a cis stereochemistry. However, the access to the corresponding triple bond may be tedious. An alternative approach is the semi-hydrogenation of an allene. Despite the fact that allene are readily accessible, their semi-hydrogenation, and especially the semi-hydrogenation of terminal allene into ω-1 alkene, has been barely disclosed in the literature and suffer from medium to low stereoselectivity and regioselectivity.

So today, there is a need to develop a novel access to alkene group from the semi-hydrogenation of allene while reaching high regioselectivity and controlling the stereoselectivity.

The present invention allows the semi-hydrogenation, using molecular H₂, of an allene into the corresponding alkene wherein said process is carried out in the presence of a heterogenous catalyst while favouring the formation of the cis isomer and reaching a high regioselectivity. To the best of our knowledge, the invention's process has never been reported in the prior art, especially high selectivity and regioselectivity was never reported for mono substituted allenes of formula (I).

### DESCRIPTION OF THE INVENTION

Surprisingly, it has now been discovered that heterogenous catalyst allows the hydrogenation of an allene with high regio- and stereoselectivity.

So, a first object of the present invention is a process for the semi-hydrogenation, using molecular H₂, of an allene of formula

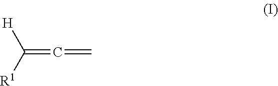

wherein R¹ is a C₂₋₃₀ hydrocarbon optionally comprising one to three oxygen atoms and/or one to two nitrogen atoms and/or one sulphur atom;
   into the corresponding alkene of formula

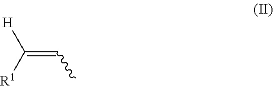

wherein R¹ have the same meaning as defined above;
   wherein said process is carried out in the presence of a heterogenous catalyst.

For the sake of clarity, by the wavy bond in compound of formula (II), or the similar, it is meant the normal meaning understood by a person skilled in the art, i.e. that the double bond may have a cis configuration corresponding to the Z isomer, a trans configuration corresponding to the E isomer or a mixture thereof. Indeed, the alkene of formula (II) may be in the form of its E or Z isomer or a mixture thereof, e.g. the invention process leads to a composition of matter consisting of one or more alkene of formula (II), having the same chemical structure but differing by the configuration of the double bond. In particular, the alkene of formula (II) can be in the form of a mixture consisting of isomers E and Z and wherein said isomer Z represents at least 50% of the total mixture, or even at least 75% (i.e a mixture Z/E comprised between 75/25 and 100/0). According to a particular embodiment the alkene of formula (II) is in a form of it Z isomer; i.e. 100% Z.

The term “optionally” is understood that a certain group to be optionally comprising can or cannot comprise a certain atom.

It is understood that by “ . . . hydrocarbon ligand . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises one two or three oxygen atoms in a form of alcohol, ketone, aldehyde, ether, ester, carboxylic acid, carbonate groups and/or one or two nitrogen atoms in a form of amine or amide groups and/or one sulfur atom in a form of a thiol group. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain. For example, a —CH₂—CH₂—CHOH—CH₂— group represents a C₄ hydrocarbon group comprising an alcohol group (substitution of a hydrogen atom), i.e. a C₄ hydrocarbon comprising an oxygen atom; a —CH₂—CH₂—COO—CH₂—CH₂CH₂—CH₂— group represents a C₇ hydrocarbon group comprising one ester group (substitution of carbon atoms/insertion into the hydrocarbon chain), i.e. a C₇ hydrocarbon comprising two oxygen atoms and, similarly, a —CH₂—CH₂—O—CH₂—CH₂—O—CH₂—CH₂— group represents a C₆ hydrocarbon group comprising two ether groups, i.e. a C₆ hydrocarbon comprising two oxygen atoms.

According to any embodiments of the inventions, when R¹ is an hydrocarbon comprising one to three oxygen atoms and/or one to two nitrogen atoms and/or one sulphur atom, said heteroatom is not directly connected to the allene functional group.

According to any one of the above embodiments of the invention, the allene of formula (I) is a C₅-C₃₀ compound of formula (I), particularly, C₅-C₂₀ compound of formula (I), particularly, C₅-C₁₅ compound of formula (I), particularly, C₅-C₁₂ compound of formula (I), even more particularly, C₈-C₁₂ compound of formula (I).

According to any one of the above embodiments of the invention, R¹ is a C₂₋₂₅ hydrocarbon optionally comprising one to three oxygen atoms. Particularly, R¹ is a C₂₋₂₀ hydrocarbon optionally comprising one to three oxygen atoms. Particularly, R¹ is a C₂₋₁₈ hydrocarbon optionally comprising one to three oxygen atoms. Particularly, R¹ is a C₃₋₁₈ hydrocarbon optionally comprising one to three oxygen atoms. Particularly, R¹ is a C₄₋₁₈ hydrocarbon optionally comprising one to three oxygen atoms.

According to any one of the above embodiments of the invention, the R¹ group is substituted by an hydroxy group. In particular, the hydroxy group is in alpha of the allene functional group. In other words, the allene of formula (I) is of formula

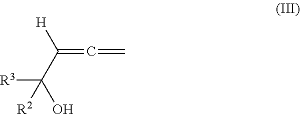

wherein R² is a C₁₋₁₄ hydrocarbon optionally comprising one to two oxygen atoms and/or one to two nitrogen atoms and/or one sulphur atom and R³ is a hydrogen atom or a C₁₋₆ hydrocarbon group; or R² and R³ are taken together and form a C₅₋₁₆ cycloalkyl or C₅₋₁₆ cycloalkenyl group, each optionally substituted by one to two hydroxy groups and/or one to three C₁₋₆ alkyl, C₂₋₆ alkenyl, C₁₋₆ alkoxy groups; and the alkene of formula (II) is of formula

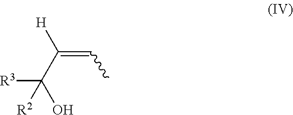

wherein R² and R³ have the same meaning as defined above.

According to any embodiments of the invention, R³ may be a hydrogen atom, a phenyl group or C₁₋₆ alkyl group. Particularly, R³ may be a hydrogen atom or C₁₋₄ alkyl group. Particularly, R³ may be a hydrogen atom or C₁₋₃ alkyl group. Particularly, R³ may be a hydrogen atom or a methyl, ethyl or isopropyl group. Particularly, R³ may be a hydrogen atom or a methyl group. Even more particularly, R³ may be a methyl group.

According to any embodiments of the invention, R² is a C₁₋₁₂ hydrocarbon optionally comprising one to two oxygen atoms. Particularly, R² is a C₁₋₁₀ hydrocarbon optionally comprising one to two oxygen atoms. Particularly, R² is a linear, branched or cyclic C₁-C₁₀ alkyl, or linear, branched or cyclic C₂-C₁₀ alkenyl group or a linear or branched C₁-C₆ phenylalkyl, each optionally substituted. Particularly, R² is a linear, branched or cyclic C₂-C₁₀ alkyl, or linear, branched or cyclic C₂-C₁₀ alkenyl group or a linear or branched C₁-C₄ phenylalkyl, optionally substituted. Particularly, R² is a linear, branched or cyclic C₂-C₉ alkyl, or linear, branched or cyclic C₂-C₉ alkenyl group or a linear or branched C₁-C₄ phenylalkyl, optionally substituted. Particularly, R² is a linear, branched or cyclic C₄-C₉ alkyl, or linear, branched or cyclic C₄-C₉ alkenyl group or a linear or branched C₁-C₂ phenylalkyl, optionally substituted. Even more particularly, R² is a linear, branched or cyclic C₅-C₉ alkyl, or linear, branched or cyclic C₅-C₉ alkenyl group or a linear or branched C₁-C₂ phenylalkyl, optionally substituted.

Furthermore, according to a yet further embodiment, when R² represents an alkenyl group then the carbon-carbon double bond is not terminal and is not conjugated with allene.

Possible substituents of R² are one, two or three OR^{h}, one or two NR^{h}₂ or one S(═O)R^{h} or one SO₂R^{h} wherein R^{h} is a hydrogen atom, a halogenated C₁-C₂ group or a C₁ to C₁₀ cyclic, linear or branched alkyl, or alkenyl group, preferably a C₁ to C₄ linear or branched alkyl or alkenyl group. As other possible substituents one may also cite a group COOR^{h}.

According to any embodiments of the invention, R² and R³ are taken together and form a C₅₋₁₄ cycloalkyl or C₅₋₁₄ cycloalkenyl group, each optionally substituted by one to two hydroxy and/or one to three C₁₋₆ alkyl, C₂₋₆ alkenyl, C₁₋₆ alkoxy groups. Particularly, R² and R³ are taken together and form a C₅₋₁₂ cycloalkyl or C₅₋₁₂ cycloalkenyl group, each optionally substituted by one to two hydroxy groups and/or one to three C₁₋₆ alkyl, C₂₋₆ alkenyl, C₁₋₆ alkoxy groups. Particularly, R² and R³ are taken together and form a C₅₋₁₀ cycloalkyl or C₅₋₁₀ cycloalkenyl group, each optionally substituted by one to two hydroxy groups and/or one to three C₁₋₆ alkyl, C₂₋₆ alkenyl, C₁₋₆ alkoxy groups. Particularly, R² and R³ are taken together and form a C₅₋₈ cycloalkyl or C₅₋₈ cycloalkenyl group, each optionally substituted by one to two hydroxy groups and/or one to three C₁₋₆ alkyl, C₂₋₆ alkenyl, C₁₋₆ alkoxy groups. Particularly, R² and R³ are taken together and form a C₅₋₆ cycloalkyl or C₅₋₆ cycloalkenyl group, each optionally substituted by one to two hydroxy groups and/or one to three C₁₋₆ alkyl, C₂₋₆ alkenyl, C₁₋₆ alkoxy groups. Particularly, R² and R³ are taken together and form a C₅₋₆ cycloalkyl or C₅₋₆ cycloalkenyl group, each optionally substituted by one to two hydroxy groups and/or one to three C₁₋₄ alkyl, C₂₋₄ alkenyl, C₁₋₄ alkoxy groups. Particularly, R² and R³ are taken together and form a C₅₋₆ cycloalkyl or C₅₋₆ cycloalkenyl group, each optionally substituted by one to two hydroxy groups and/or one to three C₁₋₃ alkyl, C₂₋₃ alkenyl, C₁₋₃ alkoxy groups. Particularly, R² and R³ are taken together and form a C₅₋₆ cycloalkyl or C₅₋₆ cycloalkenyl group, each optionally substituted by one to two hydroxy groups and/or one to three C₁₋₂ alkyl, C₂₋₃ alkenyl, C₁₋₂ alkoxy groups. Even more particularly, R² and R³ are taken together and form a C₅₋₆ cycloalkyl or C₅₋₆ cycloalkenyl group, each optionally substituted by one or two methyl.

Non-limiting examples of compound of formula (I) are 4,8-dimethylnona-1,2,7-trien-4-ol, 1-(p-tolyl) buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol, 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol, (4aS,8aS)-2-(propa-1,2-dien-1-yl)decahydronaphthalen-2-ol.

According to any embodiment of the invention, the terminal allene of formula (I) can be prepared according to the general methods described in the literature.

Non-limiting examples of compound of formula (II) are (Z)-4,8-dimethylnona-2,7-dien-4-ol, (Z)-2-phenylpent-3-en-2-ol, (Z)-3-phenylhex-4-en-3-ol, (Z)-2-methyl-3-phenylhex-4-en-3-ol, 4-methyldeca-1,2-dien-4-ol, (Z)-3-methyl-1-phenylhex-4-en-3-ol, (Z)-4,4-dimethyl-1-(prop-1-en-1-yl)cyclohexan-1-ol, (Z)-1-(prop-1-en-1-yl)cyclopentan-1-ol, (Z)-1-(prop-1-en-1-yl)cycloheptan-1-ol, (Z)-2-(prop-1-en-1-yl)decahydronaphthalen-2-ol, (4aS,8aS)-2-((Z)-prop-1-en-1-yl)decahydronaphthalen-2-ol, (Z)-1-(prop-1-en-1-yl)cyclohexan-1-ol, (Z)-1-(prop-1-en-1-yl)cyclooctan-1-ol, (Z)-1-(p-tolyl) but-2-en-1-ol, (Z)-1-cyclohexylbut-2-en-1-ol, (2Z,5E)-5-methylocta-2,5-dien-4-ol, (Z)-1-phenylhex-4-en-3-ol, (6R,Z)-6,10-dimethylundeca-2,9-dien-4-ol, (Z)-undec-2-en-4-ol.

The terms “alkyl”, “alkenyl” and “alkoxy” are understood as comprising branched and linear alkyl and alkenyl groups except if indicated otherwise. The terms “alkenyl”, “cycloalkenyl” and “heterocycloalkenyl” are understood as comprising 1, 2 or 3 olefinic double bonds, preferably 1 or 2 olefinic double bonds. The terms “cycloalkyl”, “cycloalkenyl”, “heterocycloalkyl”, “heterocycloalkenyl” and “saturated or unsaturated ring” are understood as comprising a monocyclic or fused, spiro and/or bridged bicyclic or tricyclic cycloalkyl, cycloalkenyl, heterocycloalkyl, heterocycloalkenyl groups and saturated or unsaturated ring, preferably monocyclic cycloalkyl, cycloalkenyl, heterocycloalkyl and heterocycloalkenyl groups.

According to any embodiment of the invention, the heterogenous catalyst comprises palladium (Pd) or nickel (Ni) metal. In particular the heterogenous catalyst comprises palladium (Pd) or nickel (Ni) metal supported.

According to any one of the above embodiments of the invention, said palladium (Pd) or nickel (Ni) heterogeneous catalysts is supported on a carrying material.

For the sake of clarity, by carrying material it is intended a material wherein it is possible to deposit such metal and which is inert toward the hydrogen source and the substrate.

According to any one of the above embodiments of the invention, specific and non-limiting examples of carrying material is carbon, silica, calcium carbonate, barium sulphate, titanium oxide or aluminium oxide. Such supports are well known to a person skilled in the art.

The supported or not supported palladium (Pd) or nickel (Ni) are known compounds and are commercially available. A person skilled in the art is able to select the preferred kind of metal as the way that it was deposit on the support, as the proportion of metal on support material, as the form (powder, granules, pellets, extrudates, mousses . . . ) and as the surface area of the support. The amount of metal relative to the support can range between 0.05% and 25% w/w, or even between 1% and 6%, relative to the weight on the support used. Examples of suitable heterogeneous catalyst include, but are not limited to, Lindlar catalyst (Pd—Pb/CaCO₃), or Raney nickel or Pd/C; particularly, Lindlar catalyst, or Raney nickel or sulfur-modified supported Pd catalyst as described by D. Albani, M. Shahrokhi, Z. Chen, S. Mitchell, R. Hauert, N. López, J. Pérez-Ramírez in Nat. Commun. 2018, 1-11, or poly-amines modified supported Pd catalyst as described by Q. Luo, Z. Wang, Y. Chen, S. Mao, K. Wu, K. Zhang, Q. Li, G. Lv, G. Huang, H. Li, Y. Wang in ACS Appl. Mater. Interfaces 2021, 13, 27, 31775-31784.

The heterogeneous catalysts can be added into the reaction medium of the invention's process in a large range of concentrations. As non-limiting examples, one can cite as metal concentration values those ranging from 0.01 mol % to 10 mol %, relative to the total amount of substrate. Preferably, the metal concentration will be comprised between 0.02 mol % to 5 mol %, or even between 0.04 mol % to 2 mol %. It goes without saying that the optimum concentration of metal will depend, as the person skilled in the art knows, on the nature of the latter, on the nature of the substrate, if the process is run in batch or continuously, on the temperature and on the pressure of H₂ used during the process, as well as the desired time of reaction.

The heterogeneous catalysts may be recycled at the end of the invention's process. In other words, the heterogeneous catalysts may be recovered at the end of the invention's process and use several times in the invention's process.

According to any embodiment of the invention, the process of the invention is perform in absence of base or additive.

The hydrogenation reaction can be carried out in the presence or absence of a solvent. When a solvent is required or used for practical reasons, then any solvent current in hydrogenation reactions can be used for the purposes of the invention. Non-limiting examples include aromatic solvents such as toluene, fluoro-benzene, trifluoro-toluene, ortho-difluoro-benzene, ortho-dichloro-benzene, chlorobenzene or xylene, hydrocarbon solvents such as hexane or cyclohexane, ethers such as tetrahydrofuran, methyltetrahydrofuran or MTBE, polar solvents such as primary or secondary alcohols such as isopropanol or ethanol, or mixtures thereof. The choice of the solvent is a function of the nature of the complex and the person skilled in the art is well able to select the solvent most convenient in each case to optimize the hydrogenation reaction.

In the hydrogenation process of the invention, the reaction can be carried out at a H₂ pressure comprised between 25 Pa and 100×10⁵ Pa (1 to 100 bars) or even more if desired. Again, a person skilled in the art is well able to adjust the pressure as a function of the catalyst load and of the dilution of the substrate in the solvent. As examples, one can cite typical pressures of 2 to 10×10⁵ Pa (2 to 10 bars).

The temperature at which the hydrogenation can be carried out is comprised between 0° C. and 80° C., more preferably in the range of between 20° C. and 50° C. Of course, a person skilled in the art is also able to select the preferred temperature as a function of the melting and boiling point of the starting and final products as well as the desired time of reaction or conversion.

The invention's process may carried out under batch or continuous conditions.

Another aspect of the invention is the use, in the semi-hydrogenation process as defined above, of compound being selected from the group consisting of 4,8-dimethylnona-1,2,7-trien-4-ol, 1-(p-tolyl) buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol, 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol, (4aS,8aS)-2-(propa-1,2-dien-1-yl)decahydronaphthalen-2-ol.

Some compound of formula (IV) is a novel compound and present a number of advantages as explained above and shown in the examples. Therefore, another object of the present invention is a compound for use in a semi-hydrogenation invention's process, wherein the compounds is selected from the group consisting of 4,8-dimethylnona-1,2,7-trien-4-ol, (E)-5-methylocta-1,2,5-trien-4-ol, (6R)-6,10-dimethylundeca-1,2,9-trien-4-ol, 2-methyl-3-phenylhexa-4,5-dien-3-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol and (4aS,8aS)-2-(propa-1,2-dien-1-yl)decahydronaphthalen-2-ol.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ_{H}=7.26 ppm, δ_{C}=77.0 ppm). The signal assignment was ensured by recording ¹H, ¹H—COSY, —NOESY, ¹³C, ¹H-HSQC and —HMBC experiments. Gas chromatography was performed on an Agilent 6850 Series equipped with a DB-1 or DB-Wax columns (10 m×0.1 mm ID, 0.1 μm film). The cobalt complexes were prepared according to literature procedures, see a) S. C. Meier, A. Holz, J. Kulenkampff, A. Schmidt, D. Kratzert, D. Himmel, D. Schmitz, E.-W. Scheidt, W. Scherer, C. Bülow, M. Timm, R. Lindblad, S. T. Akin, V. Zamudio-Bayer, B. von Issendorff, M. A. Duncan, J. T. Lau, and I. Krossing Angew. Chem. Int. Ed. 2018, 57, 9310-9314. b) C. S. MacNeil, H. Zhong, T. P. Pabst, M. Shevlin, P. J. Chirik ACS Catalysis 2022, 12, 4680-4687. c) M. R. Friedfeld, H. Zhong, R. T. Ruck, M. Shevlin, P. J. Chirik Science 2018, 360, 888-893. (R,R)-iPr-Duphos stands for (R,R)-1,2-bis(2,5-diisopropylphospholan-1-yl)benzene.

### Example 1

Hydrogenation of (rac)-4,8-dimethyl-1,2,7-nonatrien-4-ol Using Palladium Lindlar Type Catalyst in Various Solvent.

A 100 ml stainless autoclave was charged with (rac)-4,8-dimethyl-1,2,7-nonatrien-4-ol (20 g, 120.3 mmol), Lindlar catalyst (19 mg, Pd 5%, Pb 5% on CaCO₃) and the desired solvent (20 ml) as shown in Table 1. The autoclave was closed, purged with hydrogen (3×5 bar), and the solution was mechanically stirred under hydrogen (5 bar) at 60° C. during the indicated time. The autoclave was then cooled to room temperature, vented, purged with nitrogen and the reaction mixture analysed by GC (DB-Wax). The results are shown in Table 1.

TABLE 1
Hydrogenation of (rac)-4,8-dimethyl-1,2,7-nonatrien-4-ol
using palladium Lindlar type catalyst in various solvent.

| Solvent | Time (h) | Conv.¹⁾ | Z-product²⁾ |
|---|---|---|---|
| Methanol | 4.85 | >99 | 82 |
| Ethanol | 4.78 | 99.5 | 76 |
| Isopropanol | 2.6 | >99 | 83 |
| Toluene | 2 | >99 | 82 |
| Ethyl Acetate | 2.36 | 99 | 83 |
| Heptane | 1.5 | 99 | 82 |

¹⁾Conversion: 100% - % amount of starting material as measured by GC.
3) Amount of (rac)-(Z)-4,8-dimethyl-2,7-nonadien-4-ol as measured by GC.

### Example 2

Hydrogenation of (rac)-4,8-dimethyl-1,2,7-nonatrien-4-ol Using Raney Nickel Catalyst.

A 100 ml stainless autoclave was charged with (rac)-4,8-dimethyl-1,2,7-nonatrien-4-ol (20 g, 120.3 mmol), Raney Nickel catalyst (64 mg, washed 3× with iPrOH) and EtOH (20 ml). The autoclave was closed, purged with hydrogen (3×5 bar), and the solution was mechanically stirred under hydrogen (5 bar) at 60° C. during 26 hours. The autoclave was then cooled to room temperature, vented, purged with nitrogen and the reaction mixture analysed by GC (DB-Wax). Analysis showed a conversion 99.5% and the formation of (rac)-(Z)-4,8-dimethyl-2,7-nonadien-4-ol in 72%.

### Example 3

Hydrogenation of (rac)-4,8-dimethyl-1,2,7-nonatrien-4-ol Using Various Poisoned Palladium Catalysts.

A 100 ml autoclave with a glass liner was charged with a solution of (rac)-4,8-dimethyl-1,2,7-nonatrien-4-ol (20 g, 120 mmol), a poisoned palladium catalyst (40 mg for 5% Pd or 20 mg for 10% Pd) and ethanol (20 ml) as shown in Table 2. The autoclave was closed, purged with hydrogen (3×5 bar), and the solution was mechanically stirred under hydrogen (5 bar) at 60° C. After the indicated time, the autoclave was then cooled to room temperature, vented, purged with nitrogen and the reaction mixture analysed by GC (DB-Wax). The results are shown in Table 2.

TABLE 2
Hydrogenation of (rac)-4,8-dimethyl-1,2,7-nonatrien-
4-ol using various poisoned palladium catalysts.

| Metal | Met. | Water | | Time | | |
|---|---|---|---|---|---|---|
| catalysts | % | % | Poisoning agent | (h) | Conv.¹⁾ | Z-product²⁾ |
| Pd/C | 5 | 50 | Ethylene diamine | 22 | 100 | 76 |
| Pd/C | 10 | 50 | Ph₂S | 24 | 99 | 78 |

¹⁾Conversion: 100%-% amount of starting material as measured by GC.
²⁾Amount of (rac)-(Z)-4,8-dimethyl-2,7-nonadien-4-ol as measured by GC.

1. A process for the semi-hydrogenation, using molecular H₂, of an allene of formula 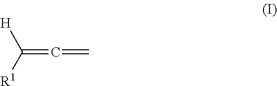
wherein R¹ is a C₂₋₃₀ hydrocarbon optionally comprising one to three oxygen atoms and/or one to two nitrogen atoms and/or one sulphur atom;
into the corresponding alkene of formula 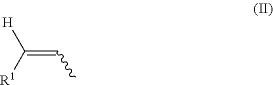
wherein R¹ have the same meaning as defined above;
wherein said process is carried out in the presence of a heterogenous catalyst.

2. The process according to claim 1, wherein the allene of formula (I) is C₅-C₁₅ compound.

3. The process according to claim 1, wherein R¹ is a C₄₋₁₈ hydrocarbon optionally comprising one to three oxygen atoms.

4. The process according to any one of claims 1 to 3, wherein the allene of formula (I) is of formula 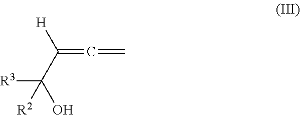
wherein R² is a C₁₋₁₄ hydrocarbon optionally comprising one to two oxygen atoms and/or one to two nitrogen atoms and/or one sulphur atom and R³ is a hydrogen atom or a C₁₋₆ hydrocarbon group; or R² and R³ are taken together and form a C₅₋₁₆ cycloalkyl or C₅₋₁₆ cycloalkenyl group, each optionally substituted by one to two hydroxy groups and/or one to three C₁₋₆ alkyl, C₂₋₆ alkenyl, C₁₋₆ alkoxy groups;
and the alkene of formula (II) is of formula 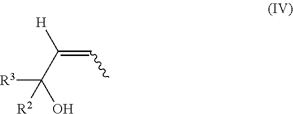
wherein R² and R³ have the same meaning as defined above.

5. The process according to claim 4, wherein R³ is a hydrogen atom or C₁₋₃ alkyl group, preferably, a hydrogen atom or a methyl, ethyl or isopropyl group.

6. The process according to any one of claims 4 to 5, wherein R² is a linear, branched or cyclic C₁-C₁₀ alkyl, or linear, branched or cyclic C₂-C₁₀ alkenyl group or a linear or branched C₁-C₆ phenylalkyl, each optionally substituted.

7. The process according to claim 4, wherein R² and R³ are taken together and form a C₅₋₆ cycloalkyl or C₅₋₆ cycloalkenyl group, each optionally substituted by one to two hydroxy groups and/or one to three C₁₋₃ alkyl, C₂₋₃ alkenyl, C₁₋₃ alkoxy groups.

8. The process according to any one of claims 1 to 7, wherein the compound of formula (I) is selected from the group consisting of 4,8-dimethylnona-1,2,7-trien-4-ol, 1-(p-tolyl) buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol, 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol and (4aS,8aS)-2-(propa-1,2-dien-1-yl)decahydronaphthalen-2-ol.

9. The process according to any one of claims 1 to 8, wherein the compound of formula (II) is selected from the group consisting of (Z)-4,8-dimethylnona-2,7-dien-4-ol, (Z)-2-phenylpent-3-en-2-ol, (Z)-3-phenylhex-4-en-3-ol, (Z)-2-methyl-3-phenylhex-4-en-3-ol, 4-methyldeca-1,2-dien-4-ol, (Z)-3-methyl-1-phenylhex-4-en-3-ol, (Z)-4,4-dimethyl-1-(prop-1-en-1-yl)cyclohexan-1-ol, (Z)-1-(prop-1-en-1-yl)cyclopentan-1-ol, (Z)-1-(prop-1-en-1-yl)cycloheptan-1-ol, (Z)-2-(prop-1-en-1-yl)decahydronaphthalen-2-ol, (4aS,8aS)-2-((Z)-prop-1-en-1-yl)decahydronaphthalen-2-ol, (Z)-1-(prop-1-en-1-yl)cyclohexan-1-ol, (Z)-1-(prop-1-en-1-yl)cyclooctan-1-ol, (Z)-1-(p-tolyl) but-2-en-1-ol, (Z)-1-cyclohexylbut-2-en-1-ol, (2Z,5E)-5-methylocta-2,5-dien-4-ol, (Z)-1-phenylhex-4-en-3-ol, (6R,Z)-6,10-dimethylundeca-2,9-dien-4-ol and (Z)-undec-2-en-4-ol.

10. The process according to any one of claims 1 to 9, wherein the heterogeneous catalyst is a Lindlar catalyst, or Raney nickel or sulfur-modified supported Pd catalyst or poly-amines modified supported Pd catalyst.

11. The process according to any one of claims 1 to 10, wherein the heterogeneous catalyst is a Lindlar catalyst or Raney nickel.

12. The process according to any one of claims 1 to 11, wherein the process of the invention is perform in absence of base or additive.

13. Use in the semi-hydrogenation process as defined in claims 1 to 12 of compound being selected from the group consisting of 4,8-dimethylnona-1,2,7-trien-4-ol, 1-(p-tolyl) buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol, 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol and (4aS,8aS)-2-(propa-1,2-dien-1-yl)decahydronaphthalen-2-ol.

14. A compound being selected from the group consisting of 4,8-dimethylnona-1,2,7-trien-4-ol, (E)-5-methylocta-1,2,5-trien-4-ol, (6R)-6,10-dimethylundeca-1,2,9-trien-4-ol, 2-methyl-3-phenylhexa-4,5-dien-3-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol and (4aS,8aS)-2-(propa-1,2-dien-1-yl)decahydronaphthalen-2-ol.
